(19) United States
(12) Patent Application Publication
(10) Pub. No.: US 2005/0233973 A1
(43) Pub. Date: Oct. 20, 2005

(54) Tri-peptides for antidepressant applications

(76) Inventors: Albert Sattin, Los Angeles, CA (US); Albert K. Pekary, Culver City, CA (US); Robert L. Lloyd, Duluth, MN (US)

Correspondence Address: PEACOCK MYERS, P.C., P O BOX 26927, ALBUQUERQUE, NM, 87125-6927, US

(21) Appl. No.: 10/940,202

(22) Filed: Sep. 13, 2004

Related U.S. Application Data
(63) Continuation-in-part of application No. 10/922,476, filed on Aug. 18, 2004.
Application No. 10/922,476 is a continuation of application No. 10/002,878, filed on Nov. 14, 2001, now abandoned.
Application No. 10/002,878 is a continuation-in-part of application No. 09/169,657, filed on Oct. 9, 1998, now Pat. No. 6,475,989.
(60) Provisional application No. 60/062,142, filed on Oct. 9, 1997.

(51) Int. Cl. A61K 38/05

(52) U.S. Cl. 514/18

(57) ABSTRACT

Provided are methods of treatment of neurological and neurobehavioral disorders, including antidepressants for use in treatment of depression and related disorders, utilizing pharmaceutical preparations including a manufactured peptide having the formula pGlu-R¹-Pro, wherein R¹ is Glu, Phe, Leu, Tyr, Lys or Val, and preferably Gly, Lys, Leu or Val, or pharmaceutically acceptable salts thereof, and a pharmaceutically acceptable carrier.

### CROSS-REFERENCE TO RELATED APPLICATIONS

This application is a continuation-in-part application of U.S. patent application Ser. No. 10/922,476 entitled Tri-Peptides for Neurological and Neurobehavior Applications, to Albert Sattin, Albert E. Pekary and Robert L. Lloyd, filed on Aug. 18, 2004, which in turn is a continuation application of U.S. patent application Ser. No. 10/002,878, entitled Tri-Peptides for Neurological and Neurobehavior Applications, to Albert Sattin, Albert E. Pekary and Robert L. Lloyd, filed on Nov. 14, 2001, which in turn is a continuation-in-part application of U.S. patent application Ser. No. 09/169,657, now U.S. Pat. No. 6,475,989 B1, entitled Use of Pyroglutamyl-Glutamyl-Prolyl Amide (EEP) for Neurological and Neurobehavioral Disorders, to Albert Sattin, Albert E. Pekary and Robert L. Lloyd, filed on Oct. 9, 1998, and issued on Nov. 5, 2002, which application claims the benefit of the filing of U.S. Provisional Patent Application Ser. No. 60/062,142, entitled Method of Treating, Diagnosing and Clinically Assessing Neurobehavior Disorders With Pyroglutamyl-Glutamyl-Prolyl Amide (EEP), Its Chemical Derivatives and Functionally Related Peptides, filed on Oct. 9, 1997, and the specification thereof of each is incorporated herein by reference.

### BACKGROUND OF THE INVENTION

1. Field of the Invention (Technical Field)

The present invention relates to tri-peptides of the general formula pGlu-X-Pro, including pGlu-X-Pro-NH₂, wherein X is Val, Leu, Tyr, Phe, Glu, Lys or an analog, isomer or mimetic of the foregoing; to peptide, peptidomimetic and protein analogs of pGlu-X-Pro, including pGlu-X-Pro-NH₂; and to methods of treating neurological and neurobehavioral conditions utilizing pGlu-X-Pro, including pGlu-X-Pro-NH₂.

2. Background Art

Note that the following discussion refers to a number of publications by author(s) and year of publication, and that due to recent publication dates certain publications are not to be considered as prior art vis-a-vis the present invention. Discussion of such publications herein is given for more complete background and is not to be construed as an admission that such publications are prior art for patentability determination purposes.

The role of small peptides in regulating various neurological and neurobehavioral pathways and conditions is the subject of significant ongoing research. One such peptide, pGlu-His-Pro-NH₂, also known as thryrotropin releasing hormone (TRH), has been studied for a number of years. TRH has been reported to alleviate depressive symptoms, but the rapid inactivation of the TRH by serum enzymes has seriously limited clinical use of this peptide. TRH is metabolized and inactivated by thyroliberinase, a blood enzyme. TRH has been shown to produce marked improvement in neurological and neuromuscular functions associated with both lower and upper neurons. See Engle, U.S. Pat. No. 4,608,365. For example, when given to patients with amyotrophic lateral sclerosis, TRH produced a marked improvement of functions of both lower and upper neurons. However, because of the instability of TRH in the blood, the beneficial effects last from only one to 24 hours and high doses are required (0.71 mg/kg of body weight, once or twice per day) depending upon the patient. Accordingly, TRH is generally administered by continuous intravenous infusions in high doses, and only in exceptional cases is intramuscular or peroral administration considered. See Geirtz et al., U.S. Pat. No. 4,906,614.

TRH is known to bind to two receptors in the central nervous system (CNS), TRHR-1, which is found primarily within the anterior pituitary, hypothalamus and spinal cord, and TRHR-2, which is widely distributed throughout the CNS. Heuer, H. et al., J Comp Neurol 428:319-36 (2000); Gershengorm, M. C. and Osman, R., Physiol Rev 76:175-91 (1996). A variety of TRH analogs have been developed by researchers that bind to TRH receptor sites within the brain. However, binding to TRHR-1 and TRHR-2 is presumptively related to activation of such receptors, resulting in effects such as release of thyroid-stimulating hormone (TSH). This activation and release of TSH is not, however, evidently necessarily involved in the pathways and mechanisms whereby TRH produces neurological and neurobehavioral applications. Thus there is a need for drugs with neurological and neurobehavioral effects similar to that of TRH, through the same, related or different pathways or receptors, but without the effects resulting from activation of TRH receptors, such as TRHR-1 and TRHR-2. Excess activation of TRH receptors can result in hyperthyroidism and related conditions. There also remains a critical need for compositions, including peptide-based compositions, which have antidepressant, analeptic, neuroprotective, euphoric or antiamnesic effects, and preferably a combination of these effects.

### SUMMARY OF THE INVENTION (DISCLOSURE OF THE INVENTION)

In one embodiment the invention provides a pharmaceutical preparation including a manufactured peptide having the formula pGlu-R¹-Pro, wherein R¹ is Leu, Tyr, Val, or Lys or pharmaceutically acceptable salts thereof, and a pharmaceutically acceptable carrier, including peptides having the formula pGlu-Leu-Pro-NH₂, pGlu-Tyr-Pro-NH₂, pGlu-Val-Pro-NH₂, or pGlu-Lys-Pro-NH₂.

In another embodiment the invention provides a method of treatment of depression, schizophrenia or affective disorders in a mammal, the method including the steps of providing a therapeutically effective amount of a composition including a peptide or pharmaceutically acceptable salt thereof of the formula pGlu-R¹-Pro, wherein R¹ is Leu, Tyr, Val or Lys and administering the composition to the mammal. The composition of the method may include a pharmaceutically acceptable carrier, and may be administered by means of oral administration or any means of parenteral administration.

In another embodiment the invention provides a method for therapy for drug dependence in a mammal, the method including the steps of providing a therapeutically effective amount of a composition comprising a peptide or pharmaceutically acceptable salt thereof of the formula pGlu-R²-Pro, wherein R² is Glu, Phe, Leu, Tyr or Val, and administering the composition to the mammal. The composition of the method may include a pharmaceutically acceptable carrier, and may be administered by means of oral administration or any means of parenteral administration.

In another embodiment the invention provides a method for inducing analgesia in a mammal, the method including the steps of providing a therapeutically effective amount of a composition comprising a peptide or pharmaceutically acceptable salt thereof of the formula pGlu-R²-Pro, wherein R² is Glu, Phe, Leu, Tyr or Val, and administering the composition to the mammal. The composition of the method may include a pharmaceutically acceptable carrier, and may be administered by means of oral administration or any means of parenteral administration.

In another embodiment the invention provides a method for inducing analeptic stimulation in a mammal, the method including the steps of providing a therapeutically effective amount of a composition comprising a peptide or pharmaceutically acceptable salt thereof of the formula pGlu-R³-Pro, wherein R³ is Glu, Leu or Val, and administering the composition to the mammal. The composition of the method may include a pharmaceutically acceptable carrier, and may be administered by means of oral administration or any means of parenteral administration.

A primary object of the present invention is to provide pharmaceutical compositions for use in the treatment of a variety of neurologic and neurobehavioral diseases, including but not limited to neuromuscular weakness, spinal cord trauma, spasticity, schizophrenia, dementias, Alzheimer's, somatization disorders, psychosomatic disorders, functional bowel disorders, epileptic disorders, sleep disorders, and loss of motoric or cognitive functions resulting from spinal cord or brain stem injuries.

Another object of the present invention is to provide pharmaceutical compositions that have effects similar to that those of TRH, but which are not subject to in vivo enzymatic degradation as rapidly as is TRH.

A primary advantage of the present invention is the relatively high metabolic stability of the compounds of the present invention in the blood.

Another advantage of the present invention is that the compounds of the invention provide desired neurological and neurobehavioral benefits without activation of receptors specific for TRH and without release of TSH.

Another advantage of the present invention is that it maintains high levels of efficacy under different modes of administration, and may be combined with many different treatments without adverse effect.

Another major advantage of the present invention is the unprecedented rapid onset of the therapeutic effect, within one day, or within hours, in contrast to established previous treatments which, such as in the case of depression, require weeks to provide desired therapeutic effect.

Other objects, advantages and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more preferred embodiments of the invention and are not to be construed as limiting the invention. In the drawings:

FIG. 1 is a profile of EEP (pGlu-Glu-Pro-NH₂) immunoreactivity following HPLC fractionation of striatum and lateral cerebellum, wherein injected samples were prepared from pooled extracts (n=6) of (left panel—A) post-ECS striatum (three swim plus three non-swim) with the standard acetonitrile gradient superimposed, and the corresponding profile (right panel—B) form sham-ECS lateral cerebellum (six non-swim).

FIG. 2 shows the typical calcium responses to pGlu-X-Pro-NH₂ peptides where X=His (TRH), Leu, Tyr and Ser in HEK293 cells or HEK293 cells stably expressing TRHR-1 or TRHR-2. Peptides were tested at concentrations equal to the IC₅₀ values. Traces are averages from 20 to 40 individual cells.

FIG. 3 shows the calcium responses of cells expressing TRHR-1 and TRHR-2. Average increases in intracellular Ca²⁺, expressed as the 340/380 fluorescence ratio, were calculated from traces such as in FIG. 2; values give the mean and S.E.M. from 33 to 120 cells from multiple experiments, with peptides tested at concentrations equal to the IC₅₀ values or, where the IC₅₀ was too high to measure, at 50 μg/mL.

FIGS. 4A and 4B show total immobility time versus swim time in a Porsolt forced swim animal model of depression, where R² is Glu, at both a high-dose (0.5 mg/kg sc) (FIG. 4A) and low-dose (0.05 mg/kg ip) (FIG. 4B).

FIG. 5 shows total immobility time versus swim time in a Porsolt forced swim animal model of depression, where R² is Phe, at high-dose (0.5 mg/kg sc).

FIG. 6 shows total immobility time versus swim time in a Porsolt forced swim animal model of depression, where R² is Lys, at 0.05 mg/kg ip.

FIG. 7 shows total immobility time versus swim time in a Porsolt forced swim animal model of depression, where R² is Tyr, at 0.05 mg/kg ip.

### DESCRIPTION OF THE PREFERRED EMBODIMENTS (BEST MODES FOR CARRYING OUT THE INVENTION)

Definitions. Certain terms as used throughout the specification and claims are defined as follows:

The “peptides” of this invention can be a) naturally-occurring, b) produced by chemical synthesis, c) produced by recombinant DNA technology, d) produced by biochemical or enzymatic fragmentation of larger molecules, e) produced by methods resulting from a combination of methods a through d listed above, or f) produced by any other means for producing peptides.

By employing chemical synthesis, a preferred means of production, it is possible to introduce various amino acids which do not naturally occur along the chain, modify the N- or C-terminus, and the like, thereby providing for improved stability and formulation, resistance to protease degradation, and the like.

The term “peptide” as used throughout the specification and claims is intended to include any structure comprised of two or more amino acids, including chemical modifications and derivatives of amino acids. The amino acids forming all or a part of a peptide may be naturally occurring amino acids, stereoisomers and modifications of such amino acids, non-protein amino acids, post-translationally modified amino acids, enzymatically modified amino acids, constructs or structures designed to mimic amino acids, and the like, so that the term “peptide” includes pseudopeptides and peptidomimetics, including structures which have a non-peptidic backbone. A “manufactured” peptide includes a peptide produced by chemical synthesis, recombinant DNA technology, biochemical or enzymatic fragmentation of larger molecules, combinations of the foregoing or, in general, made by any other method.

The “amino acids” used in this invention, and the term as used in the specification and claims, include the known naturally occurring protein amino acids, which are referred to by both their common three letter abbreviation and single letter abbreviation. See generally Synthetic Peptides: A User's Guide, G A Grant, editor, W.H. Freeman & Co., New York, 1992, the teachings of which are incorporated herein by reference. As set forth above, the term “amino acid” also includes stereoisomers and modifications of naturally occurring protein amino acids, non-protein amino acids, post-translationally modified amino acids, enzymatically synthesized amino acids, derivatized amino acids, constructs or structures designed to mimic amino acids, and the like. Modified and unusual amino acids are described generally in Synthetic Peptides: A User's Guide, cited above.

Unless the context otherwise requires, the amino acid “Glu” includes both L- and D-isomers of glutamic acid; “Phe” includes both L- and D-isomers of phenylalanine, and modified variations thereof including homophenylalanine, para-fluoro-phenylalanine, 4-bromo-phenylalanine, 4-trifluoromethyl-phenylalanine, 4-chloro-phenylalanine, 2-chloro-phenylalanine, 2,4,-dichloro-phenylalanine, 3,4,-dichloro-phenylalanine, 3,4,-difluoro-phenylalanine, 4-iodo-phenylalanine, 3,4,-dimethoxy-phenylalanine, 4-methyl-phenylalanine and 4-nitro-phenylalanine; “Leu” includes both L- and D-isomers of leucine; “Tyr” includes both L- and D-isomers of tyrosine, and modified variations thereof including O-benzyl-tyrosine and O-(2,6 dichloro)benzyl-tyrosine; “Val” includes both L- and D-isomers of valine; “Pro” includes both L- and D-isomers of proline; “Lys” includes both L- and D-isomers of lysine; and “pGlu” includes both L- and D-isomers of pyroglutamic acid.

Peptides of the Invention. The present invention includes peptides of the formula pGlu-R²-Pro, where R² is Glu, Phe, Leu, Tyr, Val or Lys. This thus includes peptides of the formula pGlu-R²-Pro-NH₂, as well as the free acid, pGlu-R²-Pro-OH, and other similar peptides.

The invention further includes precursors, including peptide and protein precursors, of peptides of the formula pGlu-R²-Pro. Precursors include direct precursors, such as peptides of the formula pGlu-R²-Pro-Gly (SEQ ID NO:1), analogous to the TRH precursor pGlu-His-Pro-Gly (SEQ ID NO:2). Precursors also include peptide fragments containing a sequence which, on in vivo modification, results in a peptide of the formula pGlu-R²-Pro. Thus precursors include peptides such as X-Y-Gln-R²-Pro-Z, where X, Y and Z may be any amino acid, analogous to the TRH precursor Lys-Arg-Gln-His-Pro-Gly (SEQ ID NO:3). Also included in the present invention are therapeutic uses of any and all peptide and protein precursors of pGlu-R²-Pro.

The categories of conditions to be treated include, but are not limited to, the affective disorders (including mood and anxiety disorders), schizophrenia, dementias, (including Alzheimer's disease), somatization disorders, spasticity, psychosomatic disorders, including functional bowel disorders, epileptic disorders, neuromuscular weakness, and sleep disorders. Any disorder of the central nervous system that is believed to be associated with an imbalance of the neuronal circuitry described by A. Sattin, Journal ECT, 15:76-92 (1999), the teachings of which are hereby incorporated by reference, may be the object of such treatment. pGlu-R²-Pro can be used in the recovery of the motor and cognitive function following spinal cord trauma or brain stem injuries.

In an alternative embodiment, pGlu-R²-Pro is used in combination with electroconvulsive therapy (“ECT”) for the treatment of a variety of mental and behavioral disorders. At present, ECT is the most effective treatment for major depression, but prior antidepressant drugs usually are not used in conjunction with such treatment. In humans, current antidepressant drugs afford no synergistic benefit at all when given with ECT. However, the effect of pGlu-R²-Pro on ECT is distinctly different from all established antidepressant drugs. pGlu-R²-Pro acts synergistically with ECT by potentiating the beneficial effects of ECT. Like pGlu-R²-Pro, TRH is also known to mediate ECT. However, the latter must be injected directly into the spinal fluid, and because of the relatively short half-life, the effects of TRH last only 1 to 2 days.

Known peptides with antidepressant or other therapeutic neurological properties, such as TRH, are easily metabolized in the body, and thus have a very short half-life. Hence large doses are required and administration of the peptide drug is often problematic. In contrast, pGlu-R²-Pro has been shown to be effective at dose rates as low as 0.5 micrograms per kilogram, and can be administered by many different modes.

Since pGlu-R²-Pro is believed to function through a mechanism that differs from any of the established antidepressant drugs, the concomitant use of any of the antidepressant drugs is also within the scope of the use of pGlu-R²-Pro in selected cases. For example, the thyroid hormone T₃ (liothyronine) has been shown to speed up the antidepressant effects of the antidepressant drugs, or to convert a non-responder to a responder. Stern et al., Biological Psychiatry, 30:623-27 (1991), reported that 50 micrograms of T₃ given at bedtime significantly reduced the required numbers of ECTs from 12 to 7. Since pGlu-R²-Pro works cooperatively with ECT some patients will benefit from the co-administration of the thyroid hormone T₃. The potentiative interaction with thyroid hormones is of value in some cases in which the desired response is not obtained with pGlu-R²-Pro alone.

In an alternative embodiment, physiological levels of various pGlu-R²-Pro peptides are used as a preliminary chemical determination of depression or to monitor its treatment. For example, the amount of pGlu-R²-Pro peptides in blood, other body fluids, or organs, in comparison with a reference standard, can be used by the health practitioner or veterinarian for the diagnosis or treatment of neurobehavioral disorders. A series of blood determinations of pGlu-R²-Pro peptides is used to assist a physician in determining the adequacy of treatment of the condition. For example, regular checks of blood level expedite determination of the appropriate dose of pGlu-R²-Pro peptides for each individual, or determine the reason for ineffectual results.

The peptides disclosed herein can be used for both medical applications and animal husbandry or veterinary applications. Typically, the product is used in humans, but may also be used in other mammals. The term “patient” is intended to denote a mammalian individual, and is so used throughout the specification and in the claims. The primary applications of this invention involve human patients, but this invention may be applied to laboratory, farm, zoo, wildlife, pet, sport or other animals.

In general, the peptides of this invention may be synthesized by solid-phase synthesis and purified according to methods known in the art. Any of a number of well-known procedures utilizing a variety of resins and reagents may be used to prepare the compounds of this invention.

The peptides of this invention may be a free base, or be in the form of any pharmaceutically acceptable salt. Acid addition salts of the compounds of this invention are prepared in a suitable solvent from the peptide and an excess of an acid, such as hydrochloric, hydrobromic, sulfuric, phosphoric, acetic, trifluoroacetic, maleic, succinic or methanesulfonic. Where the peptides of this invention include an acidic moiety, suitable pharmaceutically acceptable salts may include alkali metal salts, such as sodium or potassium salts, or alkaline earth metal salts, such as calcium or magnesium salts.

The invention provides a pharmaceutical composition that includes a peptide of this invention and a pharmaceutically acceptable carrier. The carrier may be a liquid formulation, and is preferably a buffered, isotonic, aqueous solution. Pharmaceutically acceptable carriers also include excipients, such as diluents, carriers and the like, and additives, such as stabilizing agents, preservatives, solubilizing agents, buffers and the like. Such materials may include buffers such as phosphate, citrate, acetate and other organic acid salts; antioxidants such as ascorbic acid; monosaccharides, disaccharides and other carbohydrates including cellulose or its derivatives, glucose, mannose or dextrins; chelating agents such as EDTA; sugar alcohol such as mannitol or sorbitol; counter-ions such as sodium; and non-ionic surfactants such as polyethylene glycols. The peptide may be administered in sustained-release formulations, including polymeric substrates, hydrogels, liposomes and the like.

The peptides of this invention may be formulated by any means known in the art, including but not limited to formulation as tablets, capsules, caplets, suspensions, powders, lyophilized preparations, suppositories, ocular drops, skin patches, oral soluble formulations, sprays, aerosols and the like, and may be mixed and formulated with buffers, binders, excipients, stabilizers, anti-oxidants and other agents known in the art. Administration means may include administration through mucous membranes, buccal administration, oral administration, dermal administration, rectal administration, sublingual administration, inhalation administration, nasal administration, administration by injection, and the like. All forms of such administration other than oral administration, including various forms of injection administration hereafter described, are included within the definition of “parenteral” administration.

If a peptide of this invention is administered by injection, the injection may be intravenous, subcutaneous, intramuscular, intraperitoneal or other means known in the art. For injection or other liquid administration formulations, water containing at least one or more buffering constituents is preferred, and stabilizing agents, preservatives and solubilizing agents may also be employed.

In general, the actual quantity of peptide of this invention administered to a patient will vary between fairly wide ranges depending upon the specific peptide, the disease being treated, the mode of administration, the formulation used, and the response desired. In one embodiment, from about 0.1 μg to about 1 mg of peptide per kg of body weight is administered per day.

### INDUSTRIAL APPLICABILITY

The invention is further illustrated by the following non-limiting examples.

### EXAMPLE 1

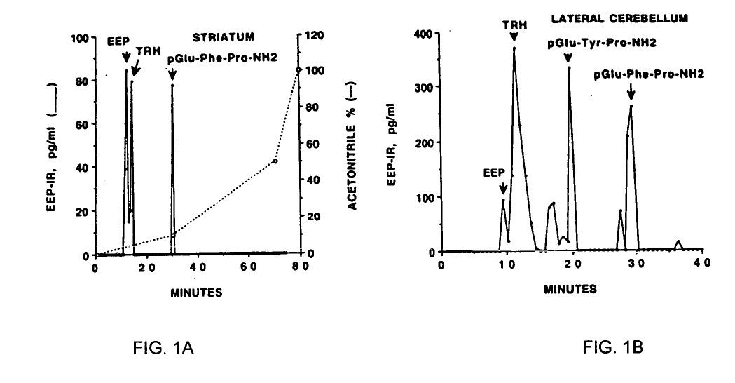

The existence of pGlu-R²-Pro, where R² is Tyr or Phe, in the brain is disclosed generally in Pekary, A. E., et al., Brain Research, 884:174-183 (2000), incorporated herein by reference. Following electroconvulsive therapy administered to male Wistar rats, using a graded increase in current doses over three stimulus days, it was found that the compound where R² is Phe increased 6-fold in the lateral cerebellum. FIG. 1 shows immunoreactivity against an antibody to pGlu-R²-Pro, where R² is Glu, following high-pressure liquid chromatography (HPLC) fractionation of striatum and lateral cerebellum subsequent to electroconvulsive therapy. The antibody to pGlu-R²-Pro, where R² is Glu, is 75% to 100% cross-reactive to the peptides where R² is Tyr or Phe.

### EXAMPLE 2

Male Wistar rats were divided into groups and received daily injections of 5 mg thyroxine (T₄), 3 mg of propylthiouracil (PTU), were castrated, or served as a control. All injected animals were treated for one week prior to sacrifice, with the last T₄ and PTU injections eighteen to twenty hours before sacrifice. After one week, the animals were sacrificed, and radioimmunoassay (RIA) procedures and co-chromatography determined levels of peptides of the formula pGlu-R²-Pro.

Table 1 shows relative potencies for displacement of assay tracer by TRH and other peptides of the general formula pGlu-R²-Pro when measured relative to the assay standard (relative potency=1.00) as determined in two TRH RIAs and one EEP (R²=Glu) RIA with the aid of a computer program. The EEP (R²=Glu) antibody (580B6) was used to RIA the HPLC fractions because it has high cross-reactivity with many pGlu-R²-Pro peptides. The 465B12 and CR-V3-B53 antibodies were raised to TRH. See generally Pekary, A. E. et al., Peptides 20:107-119 (1999); Chen Y. F. and Ramirez V. D., J. Histochem. Cytochem. 30:926-931 (1982); Pekary A. E. and Hershman J. M. (Chapter 5), in Felig P. and Frohman L. A., eds., Endocrinology and Metabolism, 4^{th} Ed., McGraw-Hill, New York, 2001. The three antibodies are not highly specific for TRH and EEP, but are very specific for peptides with the general structure pGlu-X-Pro-NH₂.

TABLE 1

| Antibody | Tracer | TRH | R² = Gln | R² = Tyr | R² = Phe | R² = Val | R² = Leu | R² = Glu |
|---|---|---|---|---|---|---|---|---|
| 465B12 | ¹²⁵I-TRH | 1.00 | 0.0225 | 9.85 | 6.79 | 0.161 | 0.301 | 0.02 |
| CR-V3-B53 | ¹²⁵I-TRH | 1.00 | 0.0055 | 3.83 | 0.738 | 0.087 | 0.088 | 0.0092 |
| 580B6 | ¹²⁵I-TRH | 2.91 | 1.00 | 5.06 | 6.53 | 9.24 | 5.00 | 1.00 |

### EXAMPLE 3

Using the methods as for Example 2, the effect of T₄, PTU and castration on HPLC peak areas corresponding to R²=Glu, Val, Tyr and Leu was determined, thereby determining the levels of pGlu-R²-Pro present in the specified tissues relative to normal controls. Peak areas are given as percentage of the corresponding HPLC peak area from the control group.

TABLE 2

| | | | | | |
|---|---|---|---|---|---|
| TRH | Group | R² = Glu | R² = Val | R² = Tyr | R² = Leu |

ACCUMBENS

| | | | | | |
|---|---|---|---|---|---|
| 128 | T₄ | 160* | 34** | 38** | 85 |
| 146* | PTU | 518** | 331** | 574** | 337** |
| 132 | Castrate | 359** | 226** | 405** | 442** |

PYRIFORM CORTEX

| | | | | | |
|---|---|---|---|---|---|
| 73 | T₄ | 88 | 22** | 14** | 101 |
| 124 | PTU | 116 | 201** | 163* | 107 |
| 226** | Castrate | 61* | 123 | 57** | 61* |

ENTORHINAL CORTEX

| | | | | | |
|---|---|---|---|---|---|
| 66* | T₄ | 43** | 53** | 54** | 119 |
| 15** | PTU | 51** | 33** | 32** | 67 |
| 4** | Castrate | 40** | 15** | 22** | 44** |

HIPPOCAMPUS

| | | | | | |
|---|---|---|---|---|---|
| 131 | T₄ | 151* | 440** | —^{a} | 149* |
| 55** | PTU | 88 | 101 | —^{a} | 71 |
| 103 | Castrate | 86 | 148* | —^{a} | 73 |

AMYGDALA

| | | | | | |
|---|---|---|---|---|---|
| 100 | T₄ | 76 | 347** | 130 | 82 |
| 75 | PTU | 38 | 54* | 76 | 73 |
| 95 | Castrate | 16** | 0** | 0** | 37** |

FRONTAL CORTEX

| | | | | | |
|---|---|---|---|---|---|
| 76 | T₄ | 93 | 127 | 41** | 68* |
| 102 | PTU | 77 | 144* | 33* | 130 |
| 200** | Castrate | 48** | 336* | 32** | 205** |

ANTERIOR CINGULATE

| | | | | | |
|---|---|---|---|---|---|
| 189** | T₄ | 94 | 324** | 25** | 97 |
| 103 | PTU | 43** | 52** | 26** | 108 |
| 237** | Castrate | 134 | 307** | 44** | 24** |

^{a}Peak areas could not be normalized because the corresponding peak in the control group was not detectable.
*p < 0.01;
**p < 0.001 by one way analysis of variance using post hoc Scheffe contrasts with the corresponding control peak area.

Marked increase in tissue levels of peptide where R²=Val or R²=Tyr in nucleus accumbens and pyriform cortex resulting from decreased levels of thyroid hormones and testosterone is consistent with either decreased release or increased synthesis of the peptides.

### EXAMPLE 4

A Porsolt swim test with rats was used to examine the antidepressant effects of peptides where R²=Glu or R²=Phe, as set forth in Lloyd, R. L. et al., Pharamcology, Biochemistry and Behavior, 70:15-22 (2001), incorporated herein by reference. The mean immobility time of animals injected with 0.5 mg/kg (sc) peptide where R²=Glu (58 seconds, S.E.=15 seconds) was significantly less (F_{1,20}=20.6, P<0.01) than the average immobility time of animals receiving saline (170 seconds, S.E.=18 seconds). No effect of the blocking variable or interaction of the blocking variable with treatment was found. In addition, an analysis of the T₃ levels of the EEP-injected (0.66 ng/mL, S.E.=0.04) and saline-injected (0.58 ng/mL, S.E.=0.03) animals failed to find a significant difference. Tissue analysis failed to find any changes in TRH or EEP in any of the brain regions analyzed.

The difference in mean immobility time for the 0.5 mg/kg (sc) of peptide where R²=Phe (125 seconds, S.E.=20 seconds) and saline-injected (188 seconds, S.E.=26 seconds) animals was smaller. As with the EEP experiment, there was no blocking effect and no interaction of the blocking variable with the treatment variable. Nevertheless, a significant (P<0.028, one-tailed) increase in the level of T₃ was found in the EEP-injected animals (0.85 ng/mL, S.E.=0.03) as compared to controls (0.77 ng/mL, S.E.=0.03). Tissue analysis failed to find any changes in TRH or EEP in any of the brain regions analyzed.

The mean immobility time of animals injected with 0.05 mg/kg (ip) EEP (121 seconds, S.E.=18 seconds) was significantly less (F_{1,18}=15.8, P<0.001) than the average immobility time of animals receiving saline (208 s, S.E.=11 s). No effect of the blocking variable or interaction of the blocking variable with treatment was found.

### EXAMPLE 5

The binding of peptides of the formula Glu-R²-Pro to TRH receptor-1 (TRHR-1) and TRH receptor-2 (TRHR-2) was evaluated. HEK293 cells stably expressing these receptors were used, with cells expressing TRHR-1 as described in Shupnik, M. A. et al., Molecular Endocrinology, 10:90-99 (1996), and cells expressing TRHR-2 prepared by transfecting HEK293 cells with a plasmid encoding TRHR-2 in pcDNA3 using lipofectamine and selecting with 0.5 mg/mL 6418 as described in Yu, R. and Hinkle, P. M., Journal Biological Chemistry, 272:28301-07 (1997).

Peptides were tested for affinity for either TRHR-1 or TRHR-2 in stably transfected HEK293 cells. Cells on 10 cm dishes were rinsed twice with 0.15 M NaCl and then scraped gently into 0.5-2 mL HBBS buffered with 15 mM HEPES to pH 7.4. Aliquots (100 μL) of suspended cells were incubated with 2 nM [³H]TRH and different concentrations of peptides at room temperature for 45 minutes. The reaction mixtures were diluted with 2.5 mL ice-cold buffered HBSS and filtered through 2.5 cm Whatman GF/A glass fiber filters, which were rinsed 3 times with 2.5 mL cold HBSS, dried, and counted. Each peptide was tested in at least 3 different experiments. Maximal binding was between 1500 and 36,000 dpm.

The density of receptors was calculated to be 0.30 pmol/mg protein for cells expressing TRHR-1 and 0.13 pmol/mg protein for cells expressing TRHR-2. Peptides were tested for the ability to compete with [³H]TRH for binding to cells expressing either receptor. There was essentially no binding of [³H]TRH to HEK293 cells not transfected with any receptor. Average IC₅₀ values are shown in Table 3. Most analogs bound TRHR-2 with slightly higher affinity than TRHR-1, but none of the peptides had high affinity for either receptor. The peptides containing Leu and Phe in the 2-position were the most potent, with affinities between 0.1 and 1% that of TRH. The Ser and Gln peptides also had measurable affinities, whereas other peptides, including R²=Thr, did not. Because [³H]TRH was used at a very low concentration, the IC₅₀ values were close to the Kᵢ values based on the equation Kᵢ=IC₅₀/(1+[L*]/K_{d(L*)}) where [L*] and K_{d(L*)} are the concentration of radioligand L* and the K_{d} for L*, respectively.

TABLE 3

| | | |
|---|---|---|
| pGlu-R²-Pro-NH₂ | TRHR-1 | TRHR-2 |

| | |
|---|---|
| R2 = | IC₅₀, μg/mL |

| | | |
|---|---|---|
| Leu | 1.85 ± 0.60 | 0.66 ± 0.24 |
| Phe | 1.58 ± 0.68 | 1.25 ± 0.44 |
| Val | >100 | >100 |
| Tyr | >100 | >100 |
| Ser | 21.7 ± 3.3 | 12.3 ± 2.9 |
| Thr | >100 | >100 |
| Gln | 9.80 ± 1.40 | 7.57 ± 0.47 |
| Glu | >100 | >100 |
| Arg | >100 | >100 |
| His (TRH) | 0.0085 ± 0.0016 | 0.0088 ± 0.0024 |

Shown are IC₅₀ values, the concentrations of peptides causing 50% inhibition of binding, for various peptides. Values given are the mean and range or SEM of 2-6 determinations.

### EXAMPLE 6

The ability of peptides of the formula pGlu-R²-Pro to stimulate an intracellular calcium response in transfected HEK293 of Example 5 was evaluated, to determine whether the peptides behave as agonists. Cells grown on 25 mm glass coverslips were loaded at room temperature with 4 μM Fura2-AM, 0.2% bovine serum albumin and 20 μg/mL cyclosporin A for 30 minutes in buffered HBSS. The coverslip was then rinsed and placed in a Sykes-Moore chamber covered with 1 mL Ca²⁺ and Mg²⁺ free HBSS. Imaging was performed on a Nikon inverted microscope with a DAGE CCD72 camera and Geniisys intensifier system. Cells were alternately excited at 340 and 380 nm and emission was measured at 490 nm; 340/380 ratios were obtained every 1.2 seconds and analyzed using Image-1 software from Universal Imaging Corporation. After a stable baseline was obtained, peptide was added to the incubation chamber and responses were followed for at least 5 minutes. Peak responses from between 33 and 120 individual cells were averaged, and responses to TRH were measured with each set of cells as a standard. Most of the peptides caused a small increase in intracellular Ca²⁺ when added to control HEK293 cells bathed in Ca²⁺-containing medium, but with the exception of the R²=Leu peptide, none of them evoked an appreciable Ca²⁺ response in Ca²⁺-free HBSS, which was used for all experiments.

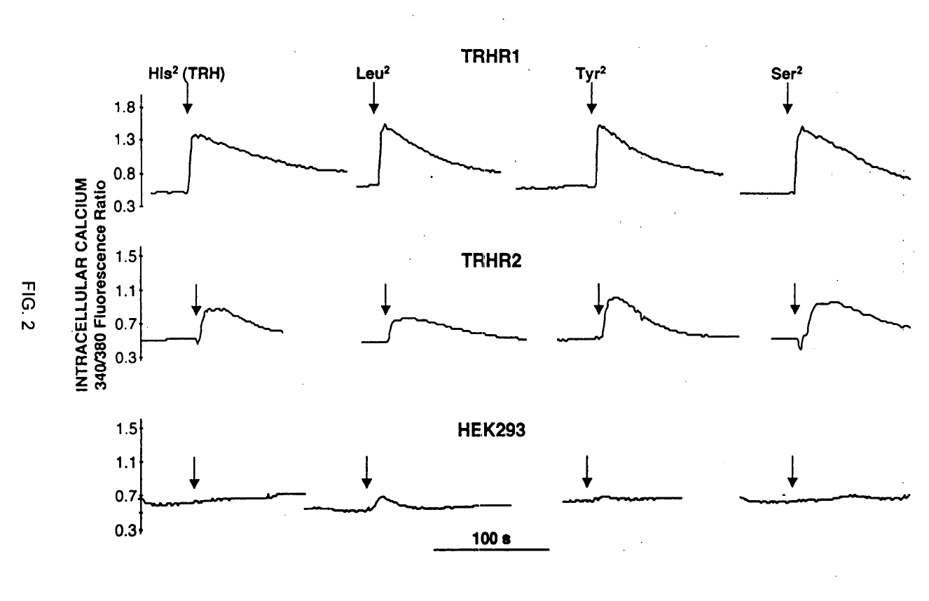

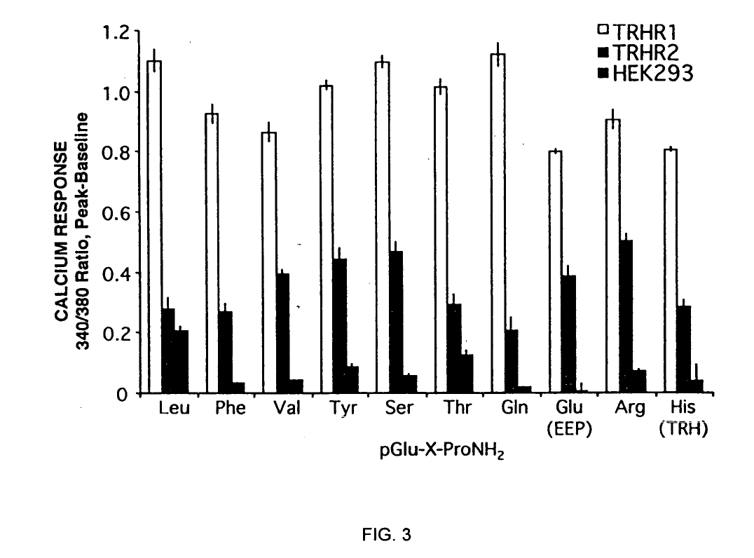

TRH is known to act via both TRHR-1 and TRHR-2 to stimulate phospholipase C and increase inositol(1,4,5)trisphosphate (IP₃), which acts on IP₃ receptors in the endoplasmic reticulum to release Ca²⁺ from intracellular stores. To determine whether the peptides were agonists or antagonists, the peptides were tested for ability to evoke a Ca²⁺ transient in control HEK293 cells or HEK293 cells expressing TRHR-1 or TRHR-2. Cells were loaded with the intracellularly trapped Ca⁺ indicator fura2 and then challenged in Ca²⁺-free buffers with peptides at concentrations equal to the calculated IC₅₀ values, where these were measurable, or at 50 μg/mL. Averaged responses from multiple individual cell traces are shown in FIG. 2. In non-transfected control HEK293 cells, TRH caused no response in HEK293 cells at concentrations up to 1 μg/mL, and pGlu-R²-Pro peptides caused little or no Ca²⁺ response except for the Leu2 peptide, which consistently introduced a small Ca²⁺ transient. In cells expressing TRHR-1 or TRHR-2, all of the peptides caused an increase in intracellular Ca²⁺. The average amplitudes are shown in FIG. 3. The Ca²⁺ transients were greater in cells expressing TRHR-1 than TRHR-2, probably reflecting the higher receptor density in this cell line. The peak heights were comparable for all of the peptides when these were tested at mid-range concentrations. In addition, the R²=Leu peptide was tested for its ability to block the Ca²⁺ response to low concentrations of TRH mediated by both receptors, and no effect was found, indicating that it does not have antagonistic activity.

### EXAMPLE 7

Groups of 10 male Wistar rats, 125 to 250 g, were anesthetized with 35 mg/kg pentobarbital. Twenty minutes after loss of the righting reflex, 10 μL of either sterile saline or peptide of this invention (1 or 10 μg/10 μL sterile saline) was administered by intracisternal (IC) injection. The time from IC injection to recovery of the righting reflex, as measured three times within 10 seconds, was recorded.

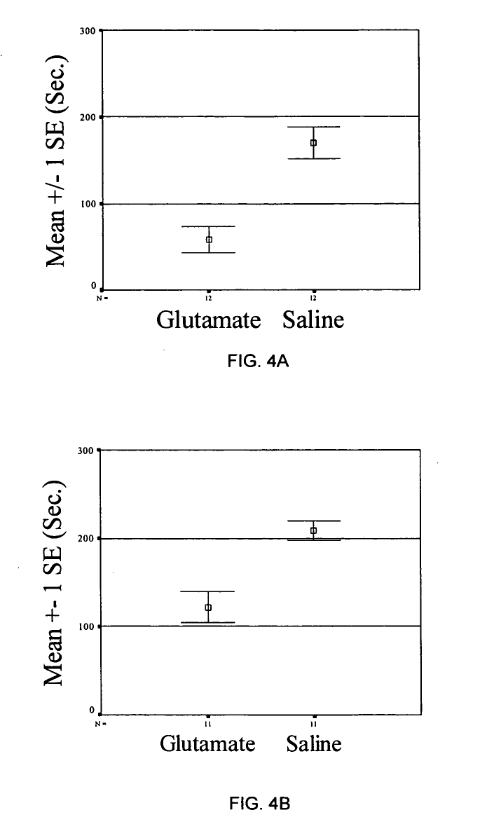

IC injection of 1 μg of TRH or pGlu-R²-Pro peptide did not result in a significant reduction in pentobarbitol-induced sleep time (analeptic effect). The results in FIG. 4 show that IC injections of 10 μg TRH and peptide where R²=Glu, Leu or Val had an analeptic effect. The injection of TRH was frequently accompanied by “wet dog” shaking while the animal was still anesthetized. R²=Glu never produced shaking while still being an effective analeptic.

### EXAMPLE 8

Levels of TRH and peptides of this invention were determined following administration, and in some instances withdrawal, of lithium. Young, adult male Sprague-Dawley rates, 300 grams at sacrifice, were divided into 4 lithium treatment groups. Control animals received a standard laboratory rodent chow. The acute group received a single IP injection of 1.5 milli-equivalents of LiCl 2 hours prior to sacrifice. The chronic and withdrawal groups received standard rodent chow containing 1.7 mg/kg LiCl for 2 weeks as well as a supplemental water bottle containing 1.45 M NaCl to compensate for the effect of LiCl on kidney function. Withdrawal rats were returned to standard chow 48 hours prior to sacrifice while the chronic animals continued on the LiCl diet. TRH, TRH-Gly (pGlu-His-Pro-Gly, a TRH precursor) (SEQ ID NO:2), EEP (pGlu-Glu-Pro-NH₂) and Ps4 (a prepro-TRH-derived TRH-enhancing decapeptide) immunoreactivity (IR) were measured in 13 brain regions. The remaining samples were pooled and fractionated by HPLC followed by EEP RIA. The most widespread changes in TRH and TRH-related peptide levels were observed in the withdrawal group compared to the controls. The direction of change for the total immunoreactivity was consistent for all TRH-IR and pGlu-R²-Pro-IR within a given tissue. For example, withdrawal increased all peptide levels in the pyriform cortex and striatum but decreased these levels in the anterior cingulate and lateral cerebellum. Both acute injection and chronic treatment with LiCl decreased TRH and TRH-related peptide levels in the entorhinal cortex. Acute injection and withdrawal both increased EEP-IR in striatum by more than 2-fold. The acute effects are most likely due to changes in the release of these peptides since 2 hours is not sufficient time for alterations in peptide biosynthesis. Chronic treatment increased levels of pGlu-Phe-Pro-NH₂ levels in the hippocampus, pGlu-Leu-Pro-NH₂, and peak “2” in the septum by more than 4-fold. The present results are consistent with a mediating role for TRH and related peptides in the mood-altering effects of lithium administration and withdrawal frequently observed during treatment for unipolar and bipolar depression.

Results are shown at Table 4 below. “AC” is acute treatment with lithium, “CHR” is chronic treatment and “WD” is withdrawal from chronic lithium treatment, with measures as the percentage of the corresponding HPLC peak area from the control group.

TABLE 4

R² =

| | | | | |
|---|---|---|---|---|
| TRH | Glu | Val | Leu | Phe |

HIPPOCAMPUS

| | | | | |
|---|---|---|---|---|
| AC | 172* | 139 | 156* | 88 |
| CHR | 90 | 72* | 96 | 433** |
| WD | 144* | 143* | 139 | 271** |

ENTORHINAL CORTEX

| | | | | |
|---|---|---|---|---|
| AC | 70* | 44** | 50** | 56* |
| CHR | 77 | 65* | 53* | 71 |
| WD | 85 | 68 | 62* | 41** |

LATERAL CEREBELLUM

| | | | | | |
|---|---|---|---|---|---|
| 109 | AC | 128 | 60* | 138 | 150* |
| 98 | CHR | 147 | 30** | 436** | 56* |
| 103 | WD | 50** | 37** | 94 | 109 |

SEPTUM

| | | | | | |
|---|---|---|---|---|---|
| 110 | AC | 124 | 93 | 150* | 41** |
| 160* | CHR | 0** | 51* | 156* | 64* |
| 122 | WD | 110 | 101 | 273** | 132 |

ACCUMBENS

| | | | | | |
|---|---|---|---|---|---|
| 43** | AC | 260** | 69 | 74 | 45** |
| 150* | CHR | 196* | 64* | 116 | 7** |
| 76 | WD | 22** | 25** | 95 | 78 |

ANTERIOR CINGULATE

| | | | | | |
|---|---|---|---|---|---|
| 18** | AC | 77 | 94 | 96 | 39** |
| 64* | CHR | 60* | 105 | 81 | 77 |
| 33** | WD | 29** | 69 | 44** | 26** |

*p < 0.01;
**p < 0.001 by one way analysis of variance using post hoc Scheffe contrasts with the corresponding control peak data.

### EXAMPLE 9

Young adult male Sprague-Dawley rats (5/group, 250 g body weight at sacrifice) were treated for 2 weeks with either twice daily injections of saline (control group), twice daily injections of 15 mg/kg cocaine until sacrifice (chronic group), single injections of 15 mg/kg cocaine 2 hours prior to sacrifice (acute group) or chronic cocaine injections replaced by saline injections 72 hours prior to sacrifice (withdrawal group). Acute cocaine produced a 4.1-fold increase in R²=Val peptide level in the medulla while R²=Val and R²=Tyr peptide increased 6.2 and 2.9-fold, respectively, in the pyriform cortex. TRH and R²=Leu peptide decreased 47% and 93%, respectively, in the nucleus accumbens while other EEP-IR peaks decreased 50% to 100%, consistent with the significant decrease in total EEP-IR in the nucleus accumbens following acute cocaine treatment. Because 2 hours is too short a time to alter levels of neuropeptides via changes in the rate of biosynthesis, the acute cocaine-induced elevation or reduction in TRH and related peptides is presumptively due to suppression or stimulation, respectively, of the corresponding peptide secretion rate.

Table 5 shows the effect of acute (AC) and chronic (CHR) treatment with cocaine and 72 hour withdrawal (WD) from chronic cocaine treatment on HPLC peak areas corresponding to R²=Glu, Val, Tyr, Leu and Phe. Peak areas are given as percentage of the corresponding HPLC peak area from the control group.

TABLE 5

| | R² = | | | | |
|---|---|---|---|---|---|
| TRH | Glu | Val | Tyr | Leu | Phe |

MEDULLA OBLONGATA

| | | | | | | |
|---|---|---|---|---|---|---|
| 96 | AC | 98 | 410** | 88 | 99 | —^{a} |
| 52 | CHR | 77 | 107 | 39* | 60 | —^{a} |
| 106 | WD | 93 | 260** | 132 | 183* | —^{a} |

PYRIFORM CORTEX

| | | | | | | |
|---|---|---|---|---|---|---|
| 67 | AC | 112 | 618** | 289** | 99 | 273** |
| 67 | CHR | 383** | 1355** | 55 | 91 | 179* |
| 89 | WD | 198** | 133 | 331** | 144* | 75* |

ENTORHINAL CORTEX

| | | | | | | |
|---|---|---|---|---|---|---|
| 70 | AC | 127 | 105 | 116 | 71 | 20** |
| 65 | CHR | 39* | 9** | 0** | 45* | 0** |
| 84 | WD | 30* | 25** | 84 | 125 | 48** |

HIPPOCAMPUS

| | | | | | | |
|---|---|---|---|---|---|---|
| 51* | AC | 70* | 29* | 110 | 29* | 194* |
| 18** | CHR | 20** | 12** | 24** | 18** | 20** |
| 108 | WD | 133 | 38* | 299** | 54 | 104 |

ANTERIOR CINGULATE

| | | | | | | |
|---|---|---|---|---|---|---|
| 57 | AC | 37* | 60 | 75 | 113 | 233* |
| 81 | CHR | 44* | 85 | 52* | 96 | 54 |
| 30* | WD | 24** | 30* | 0** | 57 | 718** |

AMYGDALA

| | | | | | | |
|---|---|---|---|---|---|---|
| 138 | AC | 44* | 79 | 58 | 48 | 62 |
| 59 | CHR | 37* | 7** | 0** | 24** | 0** |
| 58 | WD | 65 | 64 | 30* | 64 | 96 |

STRIATUM

| | | | | | | |
|---|---|---|---|---|---|---|
| 61 | AC | 78 | 113 | —^{a} | 76 | 21** |
| 20** | CHR | 43* | 64 | —^{a} | 29* | 52 |
| 93 | WD | 88 | 75 | —^{a} | 47* | 179 |

^{a}Peak areas could not be normalized because the corresponding peak in the control group could not be detected.
*p < 0.01;
**p < 0.001 by one way ANOVA using post hoc Scheffe contrast with the corresponding control peak area.

### EXAMPLE 10

Gabapentin (GP) is an antiepileptic drug that has been utilized clinically for psychiatric disorders. The effects of acute and chronic GP are important because of evidence that GP modulates glutamatergic activity wherein TRH is a likely co-transmitter. Six control and 3 treatment groups of 5 Sprague-Dawley male rats (270 g) were used. Acute (A) treatment was 10 mg IP 2 hours before sacrifice. Chronic (C) rats received 85 mg/kg/day for 2 weeks, and a withdrawal (W) group was switched to control feed 2 days before sacrifice. Twelve regions were extracted for RIA of TRH-Gly (SEQ ID NO:2) and Ps4. TRH and pGlu-R²-Pro peptides were quantitated by a combination of HPLC and 3 RIAs for pGlu-X-Pro peptides. Significant peptide changes were noted in all brain regions studied. In anterior cingulate, Ps4-IR was decreased in C and W, and TRI-Gly-IR was increased in W. In pyriform cortex, TRH-Gly-IR was decreased in C. In hippocampus, TRH-Gly was decreased in A. In striatum, Ps4-IR was increased in C. In lateral cerebellum, Ps4-IR was decreased in W. TRH and R²=Val were decreased in the medulla and R²=Leu was decreased in frontal cortex in A. R²=Glu and R²=Val peptide were increased 8.3 and 1.5-fold, respectively; R²=Leu was decreased 66% in frontal cortex in A. All peptides were increased 2 to 17-fold in frontal cortex except R²=Glu which was suppressed to undetectability in C.

Effect of acute (A) or chronic (C) treatment with gabapentin or withdrawal from gabapentin (W) treatment on the levels of TRH and pGlu-R²-Pro peptides in 3 representative brain regions is shown in Table 6. Numbers are percentage of the corresponding peak area from the control group.

TABLE 6

| | Hippocampus | | | Frontal Cortex | | | Medulla | | |
|---|---|---|---|---|---|---|---|---|---|
| R² = | A | C | W | A | C | W | A | C | W |
| Glu | 55* | 112 | 24** | 833** | 0** | 1246** | 89 | 44* | 66 |
| His (TRH) | 43* | 86 | 41* | 107 | 1686** | 726** | 50* | 66* | 74* |
| Val | 53* | 139* | 74* | 148* | 1271** | 997** | 60* | 35** | 62* |
| Tyr | 0** | 65* | 14** | 120 | 383** | 1348** | 109 | 59* | 107 |
| Leu | 38** | 142* | 85 | 34** | 766** | 745** | 107 | 71* | 95 |
| Phe | 14** | 11** | 8** | 84 | 218** | 528** | 88 | 208** | 125 |

*p < 0.01;
**p < 0.001 by one way analysis of variance using post hoc Scheffe contrasts with the corresponding control group peak area.

### EXAMPLE 11

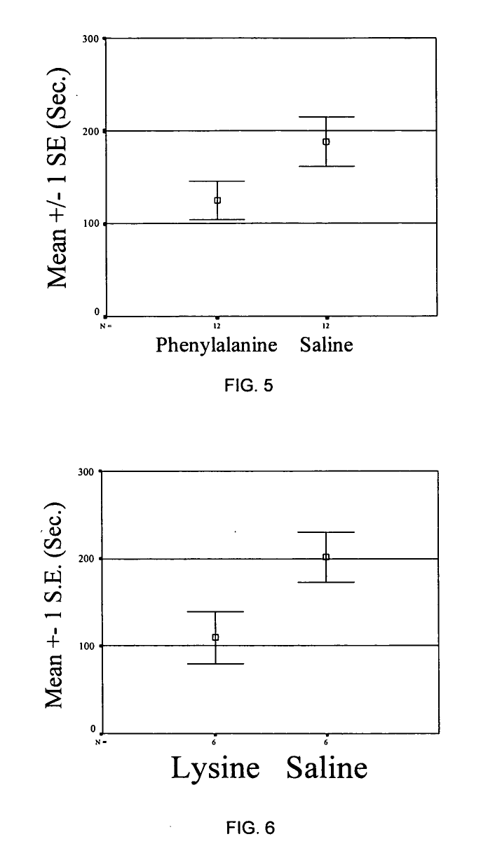

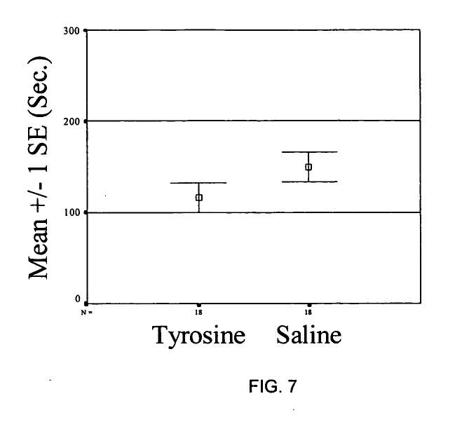

Tripeptides were made using standard synthetic methodologies where R² was Glu, Lys, Tyr, Phe, Leu and Val. A Porsolt swim test with rats was used to examine the antidepressant effects of the tripeptides as set forth in Lloyd, R. L. et al., Pharamcology, Biochemistry and Behavior, 70:15-22 (2001), incorporated herein by reference. In brief, on day 1 a 15 minute pre-swim was conducted, with a first injection immediately post-swim, and a second injection five hours later. On day two, a third injection was given 1 hour before a five minute swim test. Total immobility time versus swim time was recorded. Both high-dose (0.5 mg/kg sc) and low-dose injections (0.05 mg/kg ip) were used. At both a high-dose and low-dose, EEP (where R² is Glu) was effective, compared to saline, with a p value of less than 0.001, as shown in FIGS. 4A and 4B. At a high-dose, EFP (where R² is Phe) was effective, compared to saline, but with a p value of less than 0.086, as shown in FIG. 5. At a low-dose, EKP (where R² is Lys) was effective, compared to saline, with a p value of less than 0.042, as shown in FIG. 6. At a low-dose, ETP (where R² is Tyr) was effective, compared to saline, with a p value of less than 0.05, as shown in FIG. 7. Where R² was either Leu or Val, statistically meaningful behavioral effects were not observed in this model.

### EXAMPLE 12

Using the animal models of Example 11, T₃ (triiodothyronine) and T₄ (thyroxine) levels were tested. Serum T₃ was determined by radioimmunoassay, using fluorescence polarization of a fixed concentration of a fluorescent analog of T₃ as provided in the Abbott IM_{X} System. T₄ was also determined by radioimmunoassay. With EEP, no changes in T₃ were observed. With EKP, there was a 48% increase in T₃ (P<0.013) and a 57% increase in T₄ (p<0.009), but the increase in thyroid hormone levels did not correlate with the swim scores in the Porsolt test. With ELP (where R² is Leu) and EFP there was an increase in T₃, but this was not correlated to swim scores, with ELP not producing a behavioral effect, and EFP producing a marginal behavioral effect. No effect of EEP, ETP or EVP upon T₃ or T₄ levels was observed at the dosage used. The tripeptide EKP produced significant binding capacity in both the limbic and motor areas of the brain compared to the other TRH analogs, with the higher concentration of ELP in the striatum.

The preceding examples can be repeated with similar success by substituting the generically or specifically described reactants and/or operating conditions of this invention for those used in the preceding examples.

Although the invention has been described in detail with particular reference to these preferred embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference.

1. A method for providing therapy for drug dependence in a mammal comprising the following steps:
a) providing a therapeutically effective amount of a composition comprising a peptide or pharmaceutically acceptable salt thereof of the formula pGlu-R²-Pro, wherein R² is Glu, Phe, Leu, Tyr, Val, or Lys;
b) administering the composition to the mammal.

2. The method of claim 1 wherein the composition further comprises a pharmaceutically acceptable carrier.

3. The method of claim 1 wherein administering comprises administering by a method of administration selected from the group consisting of oral administration and parenteral administration.

4. The method of claim 1 wherein the peptide is pGlu-Glu-Pro-NH₂.

5. The method of claim 1 wherein the peptide is pGlu-Phe-Pro-NH₂.

6. The method of claim 1 wherein the peptide is pGlu-Leu-Pro-NH₂.

7. The method of claim 1 wherein the peptide is pGlu-Tyr-Pro-NH₂.

8. The method of claim 1 wherein the peptide is pGlu-Val-Pro-NH₂.

9. The method of claim 1 wherein the peptide is pGlu-Lys-Pro-NH₂

10. A method for providing analgesia in a mammal comprising the following steps:
a) providing a therapeutically effective amount of a composition comprising a peptide or pharmaceutically acceptable salt thereof of the formula pGlu-R²-Pro, wherein R² is Glu, Phe, Leu, Tyr, Val, or Lys;
b) administering the composition to the mammal.

11. The method of claim 10 wherein the composition further comprises a pharmaceutically acceptable carrier.

12. The method of claim 10 wherein administering comprises administering by a method of administration selected from the group consisting of oral administration and parenteral administration.

13. The method of claim 10 wherein the peptide is pGlu-Glu-Pro-NH₂.

14. The method of claim 10 wherein the peptide is pGlu-Phe-Pro-NH₂.

15. The method of claim 10 wherein the peptide is pGlu-Leu-Pro-NH₂.

16. The method of claim 10 wherein the peptide is pGlu-Tyr-Pro-NH₂.

17. The method of claim 10 wherein the peptide is pGlu-Val-Pro-NH₂.

18. The method of claim 10 wherein the peptide is pGlu-Lys-Pro-NH₂.

19. A pharmaceutical preparation comprising a manufactured peptide having the formula pGlu-Lys-Pro-NH₂, or pharmaceutically acceptable salt thereof, and a pharmaceutically acceptable carrier.

20. The pharmaceutical preparation of claim 19 wherein the pharmaceutically acceptable carrier comprises a buffered and isotonic carrier.


SEQUENCE LISTING (3 sequences)

SEQ ID NO: 1 - protein, 4 aa, Homo sapiens
  MISC_FEATURE (1)..(1): Xaa in position 1 is pyroglutamic acid
XXPG

SEQ ID NO: 2 - protein, 4 aa, Homo sapiens
  MISC_FEATURE (1)..(1): Xaa is pyroglutamic acid
XHPG

SEQ ID NO: 3 - protein, 6 aa, Homo sapiens
KRQHPG